(19) United States
(12) Patent Application Publication
(10) Pub. No.: US 2020/0080987 A1
(43) Pub. Date: Mar. 12, 2020

(54) BIOFLUID SENSING DEVICE CYTOKINE MEASUREMENT

(71) Applicant: Eccrine Systems, Inc., Cincinnati, OH (US)

(72) Inventors: Kathy Cunningham, Cincinnati, OH (US); Gavi Begtrup, Cincinnati, OH (US); Benjamin Katchman, Tucson, AZ (US)

(73) Assignee: Eccrine Systems, Inc., Cincinnati, OH (US)

(21) Appl. No.: 16/344,871

(22) PCT Filed: Oct. 25, 2017
(86) PCT No.: PCT/US17/58281

§ 371 Date: Apr. 25, 2019

Related U.S. Application Data
(60) Provisional application No. 62/412,351, filed on Oct. 25, 2016.

(51) Int. Cl. G01N 33/487; G16H 50/30

(52) U.S. Cl. CPC G01N 33/48785; G16H 50/30; G01N 33/48707

(57) ABSTRACT

The present disclosure provides a method of using a wearable biofluid sensing device to develop a cytokine profile for an individual. The method includes taking concentration, ratio, and trend measurements of one or more cytokines in the individual's sweat, along with other contemporaneous device measurements to inform sweat rate, skin temperature, sweat sample pH, or other factors. The method further considers these measured values in the context of external information about the individual, and uses such information to develop (1) a baseline cytokine profile characterizing the individual's healthy cytokine levels, or (2) an inflammation profile for a physiological condition, which characterizes the expected cytokine levels for a physiological condition. Also included is a method to use a biofluid sensing device to determine whether an individual has a physiological condition by comparing device measurements to the baseline profile and inflammation profile. Results are then communicated to a device user.

### CROSS-REFERENCE TO RELATED APPLICATIONS

The present application claims priority to PCT/US17/58281, filed Oct. 25, 2017, and U.S. Provisional Application No. 62/412,351, filed Oct. 25, 2016, and has specification that builds upon PCT/US16/36038, filed Jun. 6, 2016, the disclosures of which are hereby incorporated herein by reference in their entirety.

### BACKGROUND OF THE INVENTION

Among the biofluids used for physiological monitoring (e.g., sweat, blood, urine, saliva, tears), sweat has arguably the least predictable sampling rate in the absence of technological solutions. An excellent summary is provided by Sonner, et al. in the 2015 article titled “The microfluidics of the eccrine sweat gland, including biomarker partitioning, transport, and biosensing implications,” Biomicrofluidics 9, 031301, herein included by reference in its entirety. However, with proper application of technology, sweat can be made to outperform other non-invasive or less invasive biofluids in predictable sampling. In particular, sweat sensing devices hold tremendous promise for use in workplace safety, athletic, military, and clinical diagnostic settings.

A primary goal of the present invention is to provide decision support to a biofluid sensing device user that is informative at the level of the individual patient. A biofluid sensing patch worn on the skin and connected to a computer network via a reader device, such as a smart phone or other portable or stationary device, could aid in recognition of the physiological state of an individual, and relay crucial data that can inform decision making about medical treatment, physical training, safety requirements, and other applications. In certain settings, sweat sensors may continuously monitor certain aspects of an individual's physiological state and communicate relevant information to a reader device or computer network, which would then compare collected data to threshold readings and generate notification messages to the individual, a caregiver, a work supervisor, or other device user.

For example, measurement of cytokine concentrations, ratios or trends may inform a number of physiological states experienced by a device wearer. Methods to derive these physiological states and others are contemplated within the scope of the present invention.

### Definitions

Before continuing with the background, a variety of definitions should be made, these definitions gaining further appreciation and scope in the detailed description and embodiments of the present invention.

As used herein, “sweat” means a biofluid that is primarily sweat, such as eccrine or apocrine sweat, and may also include mixtures of biofluids such as sweat and blood, or sweat and interstitial fluid (ISF), so long as advective transport of the biofluid mixtures (e.g., flow) is primarily driven by sweat.

“Biofluid” means any human biofluid, including, without limitation, sweat, interstitial fluid, blood, plasma, serum, tears, and saliva.

“Biosensor” means any type of sensor that measures a state, presence, flow rate, solute concentration, solute presence, in absolute, relative, trending, or other ways in a biofluid. Biosensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a biofluid sensing device.

“Measured” can imply an exact or precise quantitative measurement and can include broader meanings such as, for example, measuring a relative amount of change of something. Measured can also imply a binary measurement, such as ‘yes’ or ‘no’ type measurements.

“Chronological assurance” means the sampling rate or sampling interval that assures measurement(s) of analytes in sample in terms of the rate at which measurements can be made of new fluid analytes as they enter the sample. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

“Analyte-specific sensor” means a sensor specific to an analyte and performs specific chemical recognition of the analytes presence or concentration (e.g., ion-selective electrodes, enzymatic sensors, electro-chemical aptamer based sensors, etc.). For example, sensors that sense impedance or conductance of a fluid, such as sweat, are excluded from the definition of “analyte-specific sensor” because sensing impedance or conductance merges measurements of all ions in sweat (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“Biofluid sensor data” means all the information collected by biofluid sensing device sensor(s) and communicated to a user or a data aggregation location.

“Correlated aggregated biofluid sensor data” means biofluid sensor data that has been collected in a data aggregation location and correlated with outside information such as time, temperature, weather, location, user profile, other biofluid sensor data, or any other relevant data.

“Sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Sensitivity” means the change in output of the sensor per unit change in the parameter being measured. The change may be constant over the range of the sensor (linear), or it may vary (nonlinear).

“Signal threshold” means the combined strength of signal-on indications produced by a plurality of aptamer sensing elements that indicates the presence of a target analyte.

“Baseline cytokine profile” means the concentrations, trends, or ratios of relevant sweat cytokines that reflect an individual's, or a group of individuals', “normal” or “healthy” level of circulating cytokines. Such a profile may include other relevant sweat sensor data, and external data.

“Inflammation profile” means the concentrations, trends, or ratios of relevant sweat cytokines that indicate an individual or group of individuals is experiencing a particular medical condition. Such a profile may include other relevant sweat sensor data, and external data.

This has served as a background for the present invention, including background technical invention needed to fully appreciate the present invention, which will now be summarized.

### SUMMARY OF THE INVENTION

The present disclosure provides a method of using a biofluid sensing device configured to be worn on an individual's skin to develop a cytokine profile for the individual. The method includes taking concentration, ratio and trend measurements of one or more cytokines in the individual's sweat, along with contemporaneous other device measurements to inform sweat rate, skin temperature, sweat sample pH, or other factors. The method further considers these measured values in the context of external information about the individual, and uses such information to develop (1) a baseline cytokine profile characterizing the individual's healthy cytokine levels, or (2) an inflammation profile for a physiological condition, which characterizes the expected cytokine levels for a physiological condition. Also included is a method to use a biofluid sensing device to determine whether an individual has a physiological condition by comparing device measurements to the baseline profile and inflammation profile. Results are then communicated to a device user.

### BRIEF DESCRIPTION OF THE DRAWINGS

The objects and advantages of the present invention will be further appreciated in light of the following detailed descriptions and drawings in which:

FIG. 1 is an example biofluid sensing device of the present disclosure.

FIG. 2 is a more detailed depiction of a biofluid sensing device of the present disclosure.

FIG. 3 is an example flow chart representing a method of indicating an individual's baseline cytokine profile in sweat.

FIG. 4 is an example flow chart representing a method of indicating a condition-specific inflammatory profile in sweat.

FIG. 5 is an example flow chart representing a method of indicating an individual's condition-specific inflammatory profile in sweat.

### DETAILED DESCRIPTION OF THE INVENTION

The detailed description of the present invention will be primarily, but not entirely, limited to devices, methods and sub-methods using wearable biofluid sensing devices. Therefore, although not described in detail here, other essential steps which are readily interpreted from or incorporated along with the present invention shall be included as part of the disclosed invention. The disclosure provides specific examples to portray inventive steps, but which will not necessarily cover all possible embodiments commonly known to those skilled in the art. For example, the specific invention will not necessarily include all obvious features needed for operation. Several specific, but non-limiting, examples can be provided as follows.

Certain embodiments of the invention show sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referenced herein by what the sensor is sensing, for example: an analyte-specific sensor; an impedance sensor such as galvanic skin response; a sweat volume sensor; a sweat generation rate sensor; and a solute generation rate sensor. Certain embodiments of the disclosed invention show sub-components of what would be biofluid sensing devices with more sub-components needed for use of the device in various applications, which are obvious (such as a battery), and for purpose of brevity and focus on inventive aspects, are not explicitly shown in the diagrams or described in the embodiments of the present disclosure.

The present disclosure applies at least to any type of biofluid sensing device that measures sweat, sweat generation rate, sweat chronological assurance, its solutes, solutes that transfer into sweat from skin, a property of or things on the surface of skin, or properties or things beneath the skin. The disclosure applies to biofluid sensing devices which can take on forms including patches, bands, straps, portions of clothing or equipment, or any suitable mechanism that reliably brings sweat stimulating, sweat collecting, and/or biofluid sensing technology into intimate proximity with sweat as it is generated. The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US15/55756, and PCT/US16/19282, each of which is included herein by reference in its entirety.

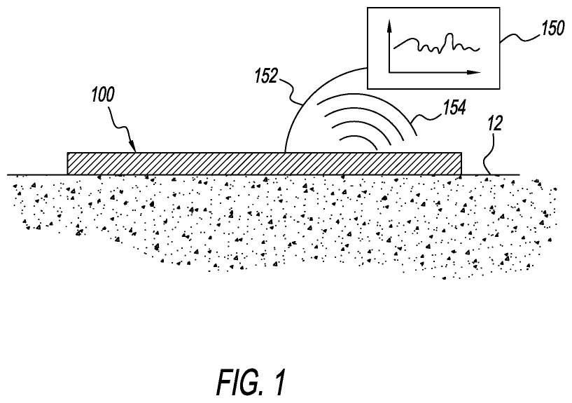

With reference to FIG. 1, a representative biofluid sensing device 100 to which the present disclosure applies is placed on or near skin 12. The biofluid sensing device may be fluidically connected to skin or regions near skin through microfluidics or other suitable techniques. Device 100 is in wired communication 152 or wireless communication 154 with a reader device 150, which could be a smart phone or portable electronic device, or for some embodiments, the device 100 and reader device 150 can be combined. Communication 152 or 154 preferably is not constant, and could be periodic or a one-time data download from device 100 once it has completed its measurements of sweat.

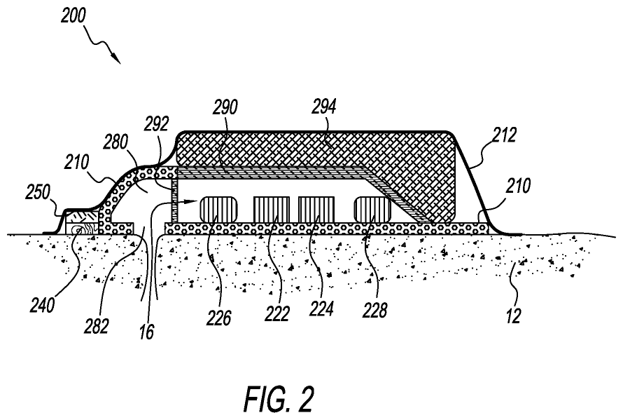

With reference to FIG. 2, a cross-sectional view of at least a portion of a biofluid sensing device capable of sweat sample concentration is provided, as disclosed in PCT/US16/58356. The device 200 includes a water-impermeable substrate 210, a protective covering 212, a microfluidic channel 280, an inlet 282 and a sweat collector (not shown) to introduce a sweat sample into the device. The substrate is constructed of PET, Kapton, or PVC, and the cover may be water impermeable rigid plastic, or in some embodiments is a breathable waterproof fabric. The channel 280 is configured to concentrate a sweat sample relative to a target analyte, and includes an optional pre-concentration filter 292, a selectively-permeable concentrator membrane 290 and a concentrator pump 294. When a sweat sample enters the channel through the inlet, it moves in the direction of the arrow 16, where it encounters the pre-filter, which could be a track-etched membrane, a cellulose triacetate filter, Dow Filmtec™, or other suitable material. The filter removes solutes from the sweat sample based on size, electrical charge, or chemical property, or removes proteases or other solutes that may interfere with the device measurements. Once through the filter, the sweat sample is concentrated relative to the target analyte by the concentrator membrane 290, which could be, for example, a dialysis membrane, or other material that at least allows the passage of water and inorganic solutes, but prevents passage of the target analyte. Depending on the application, the target analyte may be concentrated at least 10×, 100×, or 1000× higher than the unconcentrated molarity. The pump 294 is constructed of a desiccant, a wicking hydrogel, paper, fabric, or other material suitable for drawing water out of the channel through the membrane.

As the sweat sample moves through the channel, it becomes increasingly concentrated, and interacts with at least one analyte-specific sensor 222, 224 and at least one secondary sensor 226, 228. The analyte-specific sensor(s) 222, 224 are EAB or enzymatic sensors, for one or more cytokines. The secondary sensor(s) 226, 228 may include a micro-thermal flow rate sensor, one or more ISEs for measuring electrolytes (pH, Na+, Cl−, K+ Mg2+, etc.), a sweat conductivity sensor, a temperature sensor, or other sensor. Some embodiments also include a sweat stimulant gel 240 composed of sweat stimulant such as carbachol or pilocarpine, and agar, and an iontophoresis electrode 250. The electrode and stimulant gel provide iontophoretic sweat stimulation as needed. Once sweat is stimulated, the electrode 250 can also be used to measure skin impedance or galvanic skin response (“GSR”), which indicates sweat onset or sweat cessation timing.

As disclosed, a biofluid sensing device might include a plurality of other sensors to improve detection of sweat analytes, including a reference electrode, a skin temperature sensor, a GSR sensor, a skin impedance sensor, a capacitive skin proximity sensor, a heart rate monitor, a heart rate variability monitor, and an accelerometer. Many of the auxiliary features of the invention may, or may not, require other aspects of a biofluid sensing device, including two or more counter electrodes, reference electrodes, or additional supporting technology or features, which are not captured in the description herein, such as an onboard real-time clock, onboard flash memory (i.e. 1 MB minimum), Bluetooth™ or other communications hardware, and a multiplexer to process a plurality of sensor outputs.

The biofluid sensing device also includes computing and data storage capability sufficient to operate the device, which incorporates the ability to conduct communication among system components, to perform data aggregation, and to execute algorithms capable of generating notification messages. The device may have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed or cloud-based computing resources.

The biofluid sensing device's data aggregation capability may include collecting all of the sweat sensor data generated by biofluid sensing devices and communicated to the device. The aggregated biofluid sensor data could be de-identified from individual wearers, or could remain associated with an individual wearer. Such data can also be correlated with outside information, such as the time, date, medications, drug sensitivity, medical condition, activity performed by the individual, motion level, fitness level, mental and physical performance during the data collection, body orientation, the proximity to significant health events or stressors, age, sex, health history, or other relevant information. The reader device or companion transceiver can also be configured to correlate speed, location, environmental temperature or other relevant data with the sweat sensor data. The data collected could be made accessible via secure website portal to allow sweat system users to perform safety, compliance and/or care monitoring of target individuals. The biofluid sensor data monitored by the user includes real-time data, trend data, or may also include aggregated biofluid sensor data drawn from the system database and correlated to a particular user, a user profile (such as age, sex or fitness level), weather condition, activity, combined analyte profile, or other relevant metric. Trend data, such as a target individual's hydration level over time, could be used to predict future performance, or the likelihood of an impending physiological event. Such predictive capability can be enhanced by using correlated aggregated data, which would allow the user to compare an individual's historical analyte and external data profiles to a real-time situation as it progresses, or even to compare thousands of similar analyte and external data profiles from other individuals to the real-time situation. Sweat sensor data may also be used to identify wearers that are in need of additional monitoring or instruction, such as the need to drink additional water, or to adhere to a drug regimen.

Sweat is known to contain a large number of molecules that could be used to indicate an individual's physiological state. Among the most common substances found in sweat are the following: Na+, Cl−, K+, Ammonium (NH4+), urea, lactate, glucose, serine, glycerol, cortisol, and pyruvate. In addition to these common sweat analytes, each physiological condition may also have particular sweat analytes that will prove informative for indicating that physiological state. For example, blood creatinine levels have proven useful for indicating hydration levels, which also may prove true for sweat. In general, however, determining an individual's physiological state is a significant challenge. Not only is every individual different in terms of how a physiological state may present, but even a simple physiological state or disorder is a complex set of biological processes that does not readily lend itself to reduction. Consequently, a definitive diagnosis of a physiological condition often is not possible. One solution is to divide individuals according to phenotypes or susceptibilities that indicate the mode in which a physiological state is likely to manifest in those individuals. These phenotypes may be indicated by analyte signatures that emerge in sweat.

There have been relatively few studies—such as those linking sweat chloride and cystic fibrosis—examining the relationships between sweat analytes and physiological states. It is therefore necessary to build data across multiple individuals correlating physiological states with sweat analyte readings. By this means, discernable sweat analyte signatures are identified that provide useful information about a given physiological state. Further, the translation of analyte concentrations and ratios to meaningful physiological information must account for a number of variabilities unrelated to differences in concentrations. For example, sweat concentrations of analytes relative to blood or plasma concentrations are known to vary depending on sweat rate, the body location from which a sample is taken, kidney or liver disease or function, external temperatures, and other factors. Therefore, algorithms and techniques are required to adjust sweat analyte signatures to account for these variabilities.

In an embodiment of the disclosed invention, therefore, a biofluid sensing device may be configured to detect inflammatory cytokine levels in sweat or other biofluid, and use those measurements to indicate whether a device wearer is experiencing a physiological condition. Blood levels of different cytokines, such as interleukin-1α (IL-1a), vary in response to several types of conditions, including auto-immune disorders, neurological disorders, infections, cancer, and organ or joint transplants, among others. Cytokines participate in biological responses to such physiological conditions by interacting with cell surface receptors, affecting the cascade of immune cell responses, and exerting complex regulatory effects on many other cell types. Because they are involved in the initiation, expansion, and regulation of inflammatory response, cytokines may serve as leading indicators of various conditions before the conditions manifest symptomatically.

In general terms, cytokines are small proteins deployed by the immune system in response to molecular patterns that indicate infection, cellular damage or death, or genetic mismatch. Depending on the cytokine and target cell characteristics, a cytokine can promote cellular proliferation, differentiation, programmed cell death (apoptosis), or activate cells to destroy other cells. Cytokines in one area of the body can alter the gene expression and migratory behavior of nearby cells, and may exert longer-range effects that attract additional immune system response to the area. In this way, local cytokine activity can build until the resulting cytokine increase not only becomes measurable in blood-based assays, but also may influence cells in multiple locations throughout the body.

Cytokines interact with other cells through various means, including acting in a self-directed fashion (autocrine), acting on adjacent or nearby cells (paracrine), or acting on distant cells through the bloodstream (endocrine). Of these modes of interaction, the endocrine function is the most interesting for biofluid sensing device applications, since elevated concentrations of circulating cytokines can indicate a serious, organism-wide response, and such cytokines may be measured continuously or repeatedly at multiple locations on the body, such as through devices worn on the skin.

Typically, healthy individuals maintain a more or less stable complement of circulating cytokines in the bloodstream, usually in the range of 0.5 to 10 pg/mL. However, cytokines will be up-regulated in blood by as much as 1000× as the body copes with trauma, disease progression, or worsening infection. Conversely, treatment effectiveness or improvement in the condition may manifest as a reduction of cytokine levels from peak levels. Endocrine cytokine response most frequently manifests as a 2× to 10× increase in one or more cytokine levels.

Using blood tests, researchers and clinicians have developed preliminary models for using circulating inflammatory cytokines to inform and treat several categories of diseases and disorders. These models are hampered, however, by the invasive and discontinuous nature of the requisite blood samples. By contrast, sweat sensing devices promise a means to conduct non-invasive and continuous or repeated monitoring of cytokine levels and ratios, which will allow earlier detection of adverse medical trends, and will inform ongoing therapeutic decision making.

As discussed in PCT/US16/58356, and PCT/US2016/59392, each of which is incorporated herein by reference in its entirety, since cytokines are typically found in sweat in very low concentrations, biofluid sensing applications that monitor cytokines will likely require the employment of several compensatory techniques, such as sample concentration, electromagnetic shielding, advanced fluidic management, and advanced sensing technologies.

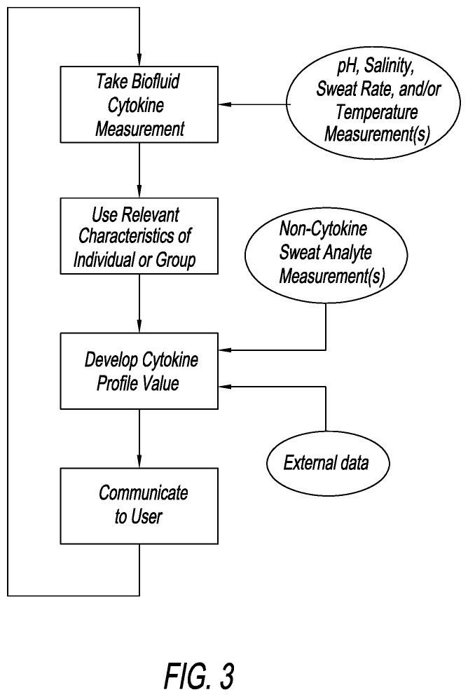

The expression of many different cytokines can be upregulated or downregulated as part of an immune response, and the amplitude and kinetics for each cytokine are highly dependent on the type and severity of the condition. Further, variations will exist in cytokine response from individual to individual. With reference to FIG. 3, an individual's “normal” or “healthy” level of circulating cytokines or individual baseline cytokine profile, will likely require some amount of external (not derived from a sweat sensor) information about the device wearer, such as age or health status, combined with baseline tracking of cytokine levels over several hours or days. In other embodiments, facts about the individual, such as age, sex, health history, fitness level, etc., may be used to select an approximate baseline cytokine profile that characterizes the individual's or group of individuals' healthy or normal cytokine levels. This baseline cytokine profile can then be compared to the biofluid sensing device measurements to develop an informed composite cytokine value.

Several cytokines are known to be present in human eccrine sweat, including IL-1a, interleukin-1β (IL-1b), interleukin-6 (IL-6), interleukin-8 (IL-8), tumor necrosis factor-α (TNFa), and transforming growth factor-β (TGFb). Further, research suggests that sweat concentrations of these cytokines correlate with their respective concentrations in plasma. See Marques-Deak, A., et al., “Measurement of cytokines in sweat patches and plasma in healthy women: Validation in a controlled study,” J. of Immunol. Methods 315 (2006) 99-109. In particular, studies indicate that interstitial fluid concentrations of IL-1b spike in reaction to a number of conditions, and therefore likely surge in sweat as well. While the aforementioned cytokines are known to exist in sweat and likely vary with plasma or ISF concentration, the disclosed invention is not limited to using only these cytokines. Other cytokines may exhibit similar properties, or may otherwise provide beneficial information for various biofluid sensing device applications, which are contemplated by the present disclosure.

Because they participate in such a variety of conditions, cytokines are non-specific indicators of physiological states. However, up-regulation and down-regulation of particular cytokines in sweat, along with information about medical context, and individual or phenotypical response variations, can be used to provide meaningful outputs for specific conditions.

A number of studies have shown that while circulating cytokine concentrations may generally increase in response to different medical or other conditions, the percentage change by each cytokine, or the ratios among the different cytokine concentrations, can inform the type of condition causing the cytokine increase. For example, burn victims typically experience a hyper-inflammatory state during which cytokines, C-reactive proteins, and other common inflammation biomarkers are elevated. See Kraft et al., “Predictive Value of IL-8 for Sepsis and Severe Infections after Burn Injury—A Clinical Study,” Shock 2015; 43(3): 222-227. However, if a burn victim's condition begins to worsen towards infection, sepsis, and ultimately death, serum levels of IL-8 and, to a lesser extent TNFa, will show further elevation. The ratios of IL-8 and TNFa to other elevated cytokines as detected by a sweat sensing device can thus be used to indicate whether a burn victim is experiencing additional health complications from their injury.

Therefore, in the context of a known condition or treatment, the up-regulation, down-regulation, or change in ratios between one or more of these cytokines, as measured by a biofluid sensing device, may prove particularly useful for applications where the non-specificity of cytokine response is suitably mitigated. These applications can be classified into at least two types: (1) acute care applications, i.e., relatively short (<30 days), critical periods that involve increased risk for a life-threatening event such as sepsis; medical implant infection; cardiovascular failure; organ damage caused by heat stress; or traumatic brain injury complications; and (2) long-term therapeutic applications, such as transplant rejection; monitoring the efficacy of mental health intervention; anti-inflammatory or immunosuppressive medication dosing; preventing dehydration; or optimizing performance (e.g., for athletes, first responders, military personnel).

Because of the generally non-specific nature of cytokine responses, many applications, including generalized infection monitoring, will benefit from information in addition to the cytokine concentration measurements themselves. This may include data from other sweat sensors, such as a cortisol EAB sensor, a dehydroepiandrosterone sulfate (DHEAS) EAB sensor, or data from other sensors, such as skin temperature, accelerometer data, heart rate, and heart rate variability. Such information may also include relevant information external to the biofluid sensing device that is correlated with the aggregated biofluid sensing data. For example, in the case of particularly weak or severely injured individuals, such as a neonate at risk for sepsis, patterns of breathing and movement may provide additional clinical insight into the individuals' condition. The sweat sensing device may therefore receive accelerometer, impedance, or microwave data as part of the overall inflammation signature to assess whether the infant's movement or breathing patterns corroborate the cytokine measurements. Likewise, external data may inform the treatment of burn victims, since the device may monitor for upregulation of IL-8, as well as increased sweat urea concentrations, and altered movement and breathing patterns. See Kraft, et al.

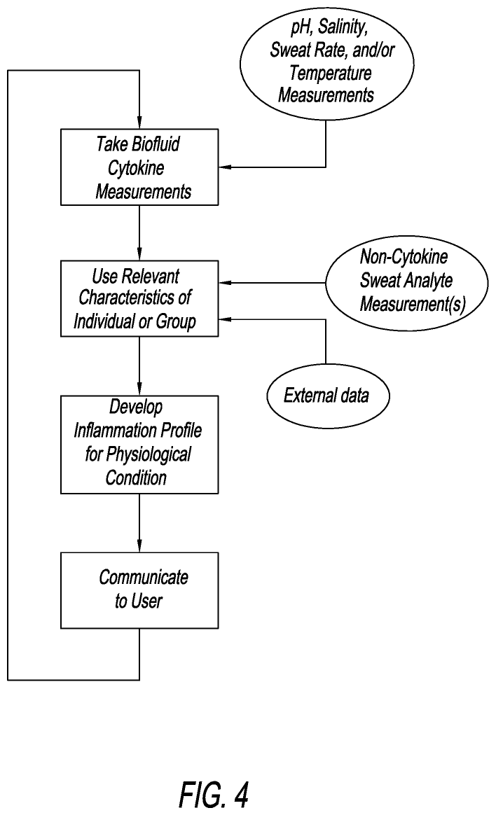

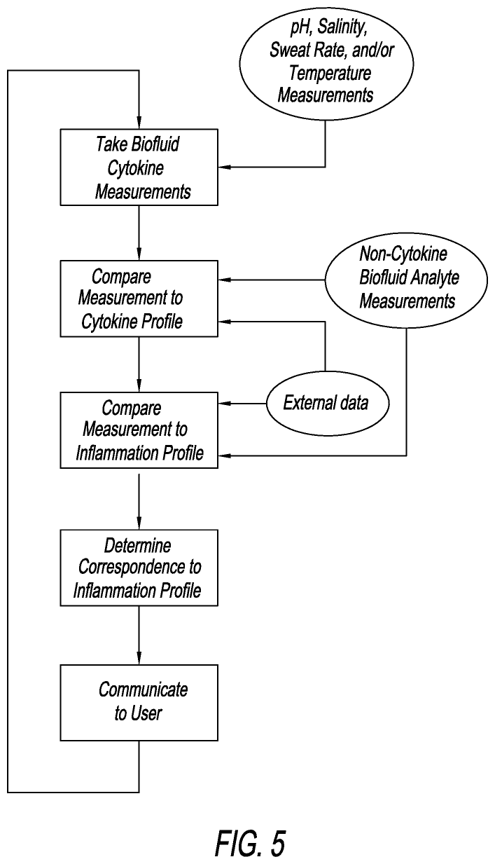

With reference to FIG. 4, to use cytokine data as part of a comprehensive profile indicative of a particular physiological condition, a biofluid sensing device can be configured to develop or use an inflammation profile that incorporates sweat concentrations, trends, and ratios of inflammatory cytokines as well as additional sensor data, and correlated aggregated data that are relevant to the physiological condition. By comparing sweat concentrations, trends, or ratios of cytokines and other sensor data in the context of an individual's baseline cytokine profile, the inflammation profile could be used to indicate, with reasonable accuracy, whether the individual was likely experiencing a particular acute care situation needing intervention, or a state requiring an adjustment to facilitate long-term therapeutic goals. See, e.g., FIG. 5.

Among exemplary acute care applications contemplated herein is sweat-based monitoring for infections with systemic involvement, such as sepsis, fungemia, viremia, and bacteremia. Blood levels of IL-1b, IL-6, IL-8 and TNFa can be variously elevated for each of these types of infection, and the scale of cytokine response, characterized through absolute concentrations, trends, and ratios among the different cytokines, can provide clinically useful information about infection progression status and severity. These increased cytokine levels can be reflected in sweat and measured by an appropriately configured biofluid sensing device. For example, neonatal sepsis is a particularly deadly condition that affects preterm and low birthweight infants. While cytokine response in neonates will manifest in upregulation of several cytokines, blood concentrations of IL-6 and TNFa increase rapidly in response to bacterial infection, and IL-8 increases proportionally to the severity of infection. See Shah and Padbury, “Neonatal sepsis—An old problem with new insights,” Virulence 2014; 5:1, 170-178. Other studies have shown that blood ratios of IL-6 can differentiate among the various types of generalized infection, including distinguishing between sepsis and fungemia, bacteremia or viremia, and IL-8 allowed distinction between bacteremia and viremia, etc. See Miring, et al., “Cytokine serum levels during post-transplant adverse events in 61 pediatric patients after hematopoietic stem cell transplantation,” BMC Cancer, 2015, Vol. 15, 607.

In addition to generalized infection monitoring, another acute care application is post-transplant/post-implant infection monitoring. Immediately after receiving an organ transplant, or a prosthetic implant, individuals are highly susceptible to developing serious infections, and their survival is heavily dependent on early and appropriate medical intervention. A suitably configured biofluid sensing device can monitor cytokine levels for indications of post-transplant infection. For example, an individual who develops an infection after receiving a hematopoietic stem cell transfusion may manifest a 5× to 10× or more increase in blood IL-6, IL-8, and TNFa. See airing, et al. Similarly, individuals receiving internal prosthetics, such as a knee, hip, or heart valve replacement, may benefit from timely detection of post-implantation infections. For example, patients receiving prosthetic heart valve replacements are susceptible to bacterial endocarditis infection, and show elevated blood concentrations of IL-6 and IL-8. See Bustamante, et al., “Cytokine profiles linked to fatal outcome in infective prosthetic valve endocarditis,” APMIS 2014; 122(6):526-9. Patients in the general population with bacterial endocarditis also showed elevated levels IL-1b and TNFa. Araújo, et al., “Cytokine Signature in Infective Endocarditis,” PLoS ONE 2015; 10(7): e0133631. Therefore, in the immediate post-transplant period, a sweat sensing device could monitor a patient's sweat concentrations or ratios of one or more of these cytokines. As another example, patients with artificial hip and knee replacements show elevated levels of IL-6 in the presence of periprosthetic joint infections. See Elgeidi, et al., “Interleukin-6 and other inflammatory markers in diagnosis of periprosthetic joint infection,” Int. Orthop. 2014; 38(12):2591-5. Recipients of organ transplants can also benefit from post-operative infection monitoring. For example, kidney transplant recipients could wear a sweat sensing device configured to monitor IL-6 and TNFa during the three months following surgery.

When building an inflammation profile for organ transplant rejection applications, certain external information can be used to improve the biofluid sensing device's predictive capabilities. For example, device applications monitoring kidney damage can monitor sweat levels of urea, creatinine, and phosphate, which will all increase with the severity of kidney damage. See Salaman, “Monitoring of rejection in renal transplantation,” Immunol. Letters 1991; 29(1-2):139-42; and Kalantar-Zadeh, et al., “Management of Minerals and Bone Disorders after Kidney Transplantation,” Curr. Opin. Nephrol. Hypertens. 2012; 21(4): 389-403. Kidney damage is also known to affect other biomarker levels, as well as blood pressure, heart rate, and heart rate variability, which could also be monitored and added to cytokine measurements in such an application. See Brotman, et al., “Heart Rate Variability Predicts ESRD and CKD-Related Hospitalization,” J. Am. Soc. Nephrol. 2010; 21(9): 1560-1570.

Other sources of inflammation, such as severe trauma, may also result in elevated cytokine levels. Therefore, other embodiments of the present disclosure may monitor sweat cytokine levels for indications of damage severity from physical trauma. For example, traumatic brain injury (TBI) is often accompanied by intracranial swelling, however, it is difficult to determine the severity of the damage, or predict whether such swelling will lead to complications from intracranial hypertension or cerebral hypoperfusion. Blood concentrations of IL-8 and TNFa have been shown to be highly predictive of TBIs that carry increased risk of such complications. A biofluid sensing device configured to measure sweat concentrations of IL-8 and TNFa may therefore provide early indication that an individual with a TBI is likely to develop serious inflammation-related complications.

In addition to the acute care applications discussed above, changes in sweat cytokine levels also allow longer-term therapeutic applications. One such application is the use of a sweat sensing device to monitor organ transplant inflammation levels. Tissue transplant rejection is a problem affecting nearly every individual receiving a tissue transplant. While some individuals reject the transplanted tissue immediately, many experience some form of acute rejection within one week, and the first three months are collectively the period of highest rejection risk. Varying levels of rejection continue for the life of the transplanted tissue. Accumulated damage from such rejection not only increases the likelihood of chronic rejection, but also leads to additional health problems, since the function of the transplanted organ deteriorates over time. Biofluid sensing applications can therefore aid patients with transplanted tissues by monitoring sweat cytokine levels for signs of acute rejection, and thereby minimizing chronic rejection damage. For example, kidney transplant patients experiencing a period of acute rejection can exhibit a 5× to 10× increase in blood levels of IL-6 and TNFa compared to patients with stable transplants. See Sonkar, et al., “Evaluation of serum interleukin 6 and tumor necrosis factor alpha levels, and their association with various non-immunological parameters in renal transplant recipients,” Singapore Med. J. 2013; 54(9): 511-515. An appropriately configured biofluid sensing device could therefore monitor the transplant patient for increased sweat levels of IL-6 and TNFa. Immunosuppressant therapies or other drugs may then be administered to counter such periods of acute rejection. The use of biofluid sensing devices may also improve long term rejection prospects by allowing physicians to fine-tune immunosuppressant or other therapies by continually monitoring and balancing inflammation levels with normal immune system function.

Another exemplary long term therapeutic application for sweat cytokine monitoring is the treatment of mental health conditions, such as chronic depression and anxiety. Cytokine levels, particularly elevated IL-6 and TNFa, have been correlated with increased risk for depression, increased severity of depression, and increased risk of suicide. See, e.g., Miller, et al., “Inflammation and Its Discontents: The Role of Cytokines in the Pathophysiology of Major Depression,” Biol. Psychiatry 2009; 65(9):732-741; Young, et al., “Is there Progress? An Overview of Selecting Biomarker Candidates for Major Depressive Disorder,” Front. Psychiatry 2016; 7:72; Hong, et al., “Pathophysiological Role of Neuroinflammation in Neurodegenerative Diseases and Psychiatric Disorders,” Int. Neurol. J. 2016; 20 Suppl. 1:S2-7; Kopschina, F., et al., “Anti-inflammatory treatment for major depressive disorder: implications for patients with an elevated immune profile and non-responders to standard antidepressant therapy,” J. Psychopharmacol. 31, 1149-1165 (2017); Miller, A., et al., “Inflammation and its discontents: the role of cytokines in the pathophysiology of major depression,” Biol. Psychiatry 65, 732-41 (2009). Similarly, successful courses of behavioral therapy and pharmaceutical intervention are able to decrease IL-6 and TNFa levels. A biofluid sensing device may therefore provide feedback to inform the effectiveness of a course of behavioral therapy, or may assist in determining the effectiveness of a particular medication to treat an individual's mental health, or could inform compliance with a drug regimen. An inflammation profile for mental health conditions could include sweat cortisol measurements to assess the individual's stress levels, in addition to relevant cytokines.

In another embodiment of the disclosed invention, a biofluid sensing device may be configured to monitor individuals with coronary artery disease (CAD)—a complex and dynamic array of heart conditions caused by defects in lipid metabolism combined with maladaptive immune responses. Coronary artery disease is an umbrella term for conditions that range in severity from stable angina, to unstable angina, to acute myocardial infarction. These latter two categories are further classified as acute coronary syndrome (ACS), and are the subject of the majority of medical interventions.

Blood levels of IL-6, TNFa, and IL-1b have been shown to be correlated with the severity of ACS, as well as CAD disease progression. For example, serum elevation of IL-6 and TNFa at the time of hospital admission are associated with morbidity and mortality prognosis across a spectrum of heart failure conditions and ACS. See Orús, et al., “Prognostic value of serum cytokines in patients with congestive heart failure,” J. Heart Lung Transplant 2000; 19(5):419-25. Similarly, the degree of IL-6 elevation in serum is associated with severity of CAD progression. See Wang, et al., “Correlation of serum high-sensitivity C-reactive protein and interleukin-6 in patients with acute coronary syndrome,” Genetics & Molecular Res. 2014; 13(2): 4260-4266 (showing that CAD progression from healthy to stable angina to unstable angina to AMI status is associated with serum IL-6 concentration increases from approximately 8 pg/mL to 11, 24 and 32, respectively). Finally, reduction in cytokines is associated with improved CAD symptoms and reduced ACS risk. See Tousoulis, et al., “Innate and Adaptive Inflammation as a Therapeutic Target in Vascular Disease—The Emerging Role of Statins,” J. Am. College Cardiol. 2014; 63:2491-502.

Physicians treating CAD have four basic objectives that may be informed by a sweat sensing device: (1) patient risk stratification, especially post-ACS or post-surgery (e.g., bypass surgery); (2) short-term patient response to ACS or surgery; (3) long-term monitoring of CAD progression; and (4) long-term monitoring of CAD response to medication or surgery.

The first two of these medical objectives may be accomplished through acute care biofluid sensing device applications. In the wake of ACS, rapid and accurate risk stratification assists the physician in choosing the most appropriate site of care, selecting therapy (e.g., medication only strategy versus early invasive intervention strategy), and estimating the patient's prognosis. A biofluid sensing device can be used to inform a patient's evaluation and risk stratification during the initial post-admission period (e.g., 24 to 72 hrs.) by taking measurements of IL-6, TNFa and IL-1b as part of a CAD Inflammation Profile. For post-ACS or post-surgery response monitoring, a device can be applied prior to the initiation of medication or surgery, and monitoring can then be maintained for as long as needed (e.g., 3 to 14 days). See Liebetrau, et al., “Release Kinetics of Inflammatory Biomarkers in a Clinical Model of Acute Myocardial Infarction,” Circ. Res. 2015; 116:867-875 (showing that controlled cardiac cell death during surgery led to a 2× increase in serum IL-6 (to 2.6 pg/mL) by 45 minutes post-procedure, and building to a 9× increase (13.6 pg/mL) by 24 hours).

Longer-term applications involve continuous or near continuous monitoring of CAD progression. One difficulty currently facing efforts to treat individuals with CAD is that much of the damage to heart tissues and arteries is caused by subclinical events that go largely unnoticed by the patient and physician. Studies indicate that as CAD progresses, the arterial plaque occlusion that triggers AMI does not develop as a singular event, but instead forms through repeated cycles of sub-clinical plaque disruption and inflammation. See Ahmadi, et al., “Do Plaques Rapidly Progress Prior to Myocardial Infarction?—The Interplay Between Plaque Vulnerability and Progression,” Circ. Res. 2015; 117:99-104. Additionally, recent reports suggest that heart attacks are clinically silent almost half (45%) of the time. See: Zhang, et aL, “Race and Sex Differences in the Incidence and Prognostic Significance of Silent Myocardial Infarction in the Atherosclerosis Risk in Communities (ARIC) Study,” Circulation 2016; 133(22):2141-8. Consequently, continuous or periodic monitoring of a patient's cytokine levels may be used to detect the timing and frequency of these repeated cycles of plaque disruption and plaque inflammation, or may detect clinically silent myocardial infarctions to provide insight for risk stratification and treatment selection. See also Reijers, J., et al. “MDCO-216 Does Not Induce Adverse Immunostimulation, in Contrast to Its Predecessor ETC-216,” Cardiovasc. Drugs Ther. 31, 381-389 (2017) (discussing TNF-α and IL-1b levels as indicative of the efficacy of cardiovscular medication regimens).

For example, a patient with stable angina who has low and stable cytokine measurements over a two-week test period may be classified as having a relatively low risk of CAD progression. By contrast, a patient with unstable angina whose cytokine measurements are generally elevated and feature frequent spikes may be selected for more aggressive medical intervention. In other uses, the cytokine measurements for an individual receiving treatment for CAD may indicate treatment effectiveness. For example, a patient receiving a new intervention, such as a new drug regimen, substantial dietary changes, smoking cessation, or initiation of an exercise program may have their cytokines monitored for improvements such as overall lower levels of cytokines, reduced IL-6 and TNFa, or relatively fewer or less severe cycles of plaque disruption or sub-clinical heart attack. The cytokine monitoring as described may also be combined with conventional approaches, such as the use of a Holter monitor or imaging studies to better inform CAD monitoring. For all biofluid sensing device applications involving CAD monitoring, the inflammation profile may also include other information, such as heart rate, blood pressure, pulse oximetry, activity level (accelerometer), and other available biomarkers, such as sweat cortisol levels.

As with long term heart disease monitoring, sweat sensing devices may also be used to monitor long-term lung disease through cytokine profiles. For example, individuals who are vulnerable to frequent lung infections, such as people with cystic fibrosis or COPD, have shown elevated levels of IL-1a, and IL-1b, along with resulting signal cascades. See Borthwick, L., “The IL-1 cytokine family and its role in inflammation and fibrosis in the lung,” Semin. Immunopathol. 38, 517-34 (2016). These cytokines or the upregulated signal molecules they trigger, therefore may be monitored to provide long-term management of such lung conditions.

In another embodiment of the disclosed invention, the biofluid sensing device may be configured to monitor for optimal physical stress thresholds. A variety of situations commonly encountered by high performance individuals (e.g., elite athletes or military members), can lead to acute or chronic elevations in cytokine levels. At the highest levels of performance, individuals may wish to train or operate at the inflection between beneficial exertion and overexertion. For such individuals, sweat cytokine measurements can inform physical stress levels that optimize training and program execution, while minimizing or avoiding negative impacts on long-term health.

Although moderate levels of exercise are beneficial, exercise performed under extreme conditions of duration, intensity, thermal environment, inadequate nutrition, sleep deprivation, or psychological stress can lead to acute increases in cytokines that are associated with increased risk of infection, injury, and decreased performance. See Nieman, “Exercise effects on systemic immunity,” Immunology & Cell Biology 2000; 78:496-501; and MacKinnon, “Overtraining effects on immunity and performance in athletes,” Immunology & Cell Biology 2000; 78:502-509.

A number of conditions associated with high physical stress manifest in the form of gastrointestinal (GI) barrier disorders that may be informed by sweat cytokines. For example, heat stress can damage the GI barrier, allowing bacterial endotoxin and other harmful GI contents to leak into the bloodstream. See Lambert, “Stress-induced gastrointestinal barrier dysfunction and its inflammatory effects,” J. Animal Sci. 2009; 87:E101-E108. Once outside of the GI tract, bacterial endotoxin triggers the upregulation of IL-1a and TNFa, and a strong inflammatory response, which can be severe enough to cause organ damage. See Bouchama, et al., “Endotoxemia and release of tumor necrosis factor and interleukin 1 alpha in acute heatstroke,” J. Appl. Physiol. 1991; 70(6):2640-4). Other studies show that endotoxemia can cause the upregulation of IL-6, TNFa, IL-10, and IL-1Ra. See Selkirk, G., et al., “Mild endotoxemia, NF-B translocation, and cytokine increase during exertional heat stress in trained and untrained individuals,” AJP Regul. Integr. Comp. Physiol. 295, R611-R623 (2008). Similar effects have been shown for extended endurance physical exertion, and accompanying psychological stress. See Jeukendrup, et al., “Relationship between gastro-intestinal complaints and endotoxemia, cytokine release and the acute-phase reaction during and after a long-distance triathlon in highly trained men,” Clin. Sci. (London) 2000; 98(1):47-55; Lambert, G., et al., “Intestinal Barrier Dysfunction, Endotoxemia, and Gastrointestinal Symptoms: The ‘Canary in the Coal Mine’ during Exercise-Heat Stress?,” Thermoregulation & Human Performance 53, 61-73 (2008); Kiers, D., et al., Characterization of a model of systemic inflammation in humans in vivo elicited by continuous infusion of endotoxin,” Sci. Rep. 7, 40149 (2017) (showing 10× increase in plasma TNFa within one hour of exposure to endotoxin).

Dehydration is also known to increase the permeability of the gastrointestinal tract, leading to the upregulation of certain cytokines. See Lambert, G., et al., “Fluid Restriction during Running Increases GI Permeability,” Int. J. Sports Med. 29, 194-198 (2008). Together with heat stress, dehydration can work to seriously undermine GI tract function, synergistically increasing gut permeability, and resulting in the upregulation of endotoxemia-related cytokines. See Sawka, M., et al., “Integrated Physiological Mechanisms of Exercise Performance, Adaptation, and Maladaptation to Heat Stress,” Comprehensive Physiology 1, 1883-1928 (2011). Sweat sensing may prove a particularly effective means for the early detection of such conditions.

Excessive endurance exertion has also been shown to result in upregulated TNFa (3× increase) and cardiac and immune system dysfunction. See La Gerche, et al., “Relationship between Inflammatory Cytokines and Indices of Cardiac Dysfunction following Intense Endurance Exercise,” PLoS ONE 2015; 10(6): e0130031. Similarly, high intensity exertion, such as that individuals may experience during periods of military training or operations, or athletic contests, have resulted in upregulated IL-6, see Gomez-Merino, et al., “Immune and hormonal changes following intense military training,” Military Medicine 2003; 168(12):1034-8, and studies show that sleep deprivation can also result in a surge of plasma TNFa. Chennaoui, et al., “Effect of one night of sleep loss on changes in tumor necrosis factor alpha (TNF-α) levels in healthy men,” Cytokine 2011; 56(2):318-24. Evidence also suggests that extreme physical exertion can result in decreased DHEA, decreased testosterone, and increased cortisol. Gomez-Merino, et al.; Brooks and Carter, “Overtraining, Exercise, and Adrenal Insufficiency,” J. Novel Physiotherapy 2013; 3(125)). An inflammation profile for physical exertion could also include sweat cortisol, testosterone, vasopressin, and DHEA in addition to relevant cytokines.

This has been a description of the present invention along with a preferred method of practicing the present invention.

What is claimed is:

1. A method of using a wearable biofluid sensing device, comprising:
taking one or more cytokine measurements of one or more cytokines in a sweat sample taken from an individual;
taking one or more secondary measurements of the sweat sample;
developing a cytokine profile based on the one or more cytokine measurements, the one or more secondary measurements, and one or more characteristics of the individual; and
communicating said cytokine profile to a device user.

2. The method of claim 1, wherein the cytokine profile is one of the following: a baseline profile, comprising a set of cytokine values representing a healthy physiological state; and an inflammation profile, comprising a set of cytokine values representing one of: a presence of inflammation, an absence of inflammation, or a severity of inflammation.

3. The method of claim 1, wherein the cytokine profile is developed for a group of persons, wherein each person in the group of persons shares one or more characteristics of the individual.

4. The method of claim 1, wherein the one or more secondary measurements is one of the following: a sweat electrolyte concentration measurement; a sweat pH measurement; a sweat salinity measurement; a skin temperature measurement; a skin impedance measurement; a galvanic skin response measurement; a sweat generation rate measurement; a heart rate measurement; a blood pressure measurement; an accelerometry measurement; and a pulse oximetry measurement.

5. The method of claim 1, wherein the one or more secondary measurements is a measurement of an analyte that is not a cytokine.

6. The method of claim 5, wherein the analyte is one of the following: Na+; K+; Cl−; NH₄+; DHEA; testosterone; vasopressin; and cortisol.

7. The method of claim 1, wherein the one or more characteristics of the individual include one or more of the following: an age; a sex; a fitness level; a medical condition; a health history; a physical activity level; and a hydration level.

8. (canceled)

9. A method of using a wearable biofluid sensing device, comprising:
taking one or more cytokine measurements of one or more cytokines in a sweat sample taken from an individual;
taking one or more secondary measurements of the sweat sample;
comparing the one or more cytokine measurements and the one or more secondary measurements to a baseline profile to develop a composite cytokine value for the individual;
comparing the composite cytokine value to an inflammation profile for a physiological state; and
communicating a result to a device user, wherein the result includes one of the following for the individual: a presence of the physiological state, a severity of the physiological state, or an absence of the physiological state.

10. The method of claim 9, wherein the physiological state is one of the following: sepsis, fungemia, viremia, and bacteremia; and wherein the one or more cytokine measurements includes a measurement of one of the following: IL-1b, IL-6, IL-8, and TNFa.

11. The method of claim 9, wherein the physiological state is one of the following: dehydration, heat stress, overexertion, and endotoxemia; and wherein the one or more cytokine measurements includes a measurement of one of the following: IL-1a, TNFa, IL-6, IL-10, and IL-1Ra.

12. The method of claim 9, wherein the physiological state is one of the following: coronary artery disease, stable angina, unstable angina, and acute myocardial infarction; and wherein the one or more cytokine measurements includes a measurement of one of the following: IL-1b, IL-6, and TNFa.

13. The method of claim 9, wherein the physiological state is one of the following: cystic fibrosis, chronic obstructive pulmonary disease (COPD), and lung inflammation; and wherein the one or more cytokine measurements includes a measurement of one of the following: IL-1a, and IL-1b.

14. The method of claim 9, wherein the physiological state is one of the following: a post-transplant infection, a post-implant infection, and an organ transplant rejection; and wherein the one or more cytokine measurements includes a measurement of one of the following: IL-6, IL-8, IL-1b, and TNFa.

15. The method of claim 9, wherein the physiological state is traumatic brain injury; and wherein the one or more cytokine measurements includes a measurement of one of the following: IL-8, and TNFa.

16. The method of claim 9, wherein the physiological state is one of the following: depression, and anxiety; and wherein the one or more cytokine measurements includes a measurement of one of the following: IL-6, and TNFa.
